SEQUENCE LISTING (18 sequences)

SEQ ID NO: 1 - DNA, 582 bp, synthetic construct
  source 1..582: mol_type = other DNA; note = Codon Optimized GM2A Sequence
atgcaaagcctgatgcaggctcctctgctgatcgccctgggactgctgctcgccgcccctgcccaggcccacctgaagaaacctagccagctgtctagctttagctgggacaattgcgacgagggcaaggaccccgccgtgatcagaagccttacactggaacctgaccctatcatcgtgcctggcaacgtgaccctgtccgtcatgggctctacaagcgttcctctgagcagccctctgaaggtggacctggtgctggagaaggaggtggccggactgtggatcaagattccttgtaccgattatatcggctcttgtaccttcgagcacttctgcgatgtgctggatatgctgatccccaccggcgagccttgcccggaacccctgagaacatacggcctgccatgccactgccccttcaaggaaggaacctactccctgcctaagagcgagttcgtggtgcccgacctggaactgccaagttggctgacaaccggcaactacagaatcgagtccgtgctgagcagcagcggcaagcggctgggctgcatcaagatcgccgcttctctgaaaggcatctga

SEQ ID NO: 2 - protein, 193 aa, synthetic construct
  source 1..193: note = GM2A Protein Sequence
MQSLMQAPLLIALGLLLAAPAQAHLKKPSQLSSFSWDNCDEGKDPAVIRSLTLEPDPIIVPGNVTLSVMGSTSVPLSSPLKVDLVLEKEVAGLWIKIPCTDYIGSCTFEHFCDVLDMLIPTGEPCPEPLRTYGLPCHCPFKEGTYSLPKSEFVVPDLELPSWLTTGNYRIESVLSSSGKRLGCIKIAASLKGI

SEQ ID NO: 3 - DNA, 3682 bp, synthetic construct
  source 1..3682: mol_type = other DNA; note = Full Sequence GM2A including pUC57 Kan
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagatggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgagcagaattcgccgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgttccggaaagccaccatgcaaagcctgatgcaggctcctctgctgatcgccctgggactgctgctcgccgcccctgcccaggcccacctgaagaaacctagccagctgtctagctttagctgggacaattgcgacgagggcaaggaccccgccgtgatcagaagccttacactggaacctgaccctatcatcgtgcctggcaacgtgaccctgtccgtcatgggctctacaagcgttcctctgagcagccctctgaaggtggacctggtgctggagaaggaggtggccggactgtggatcaagattccttgtaccgattatatcggctcttgtaccttcgagcacttctgcgatgtgctggatatgctgatccccaccggcgagccttgcccggaacccctgagaacatacggcctgccatgccactgccccttcaaggaaggaacctactccctgcctaagagcgagttcgtggtgcccgacctggaactgccaagttggctgacaaccggcaactacagaatcgagtccgtgctgagcagcagcggcaagcggctgggctgcatcaagatcgccgcttctctgaaaggcatctgagtcgacgccaataaagagctcagatgcatcgatcagagtgtgttggttttttgtgtgccactccctctctgcgcgctcgctcgctcactgaggccgggcgaccaaaggtcgcccgacgcccgggctttgcccgggcggcctcagtgagcgagcgagcgcgcagagagggaatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 4 - DNA, 161 bp, synthetic construct
  source 1..161: mol_type = other DNA; note = ITR
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtga

SEQ ID NO: 5 - DNA, 113 bp, synthetic construct
  source 1..113: mol_type = other DNA; note = Truncated ITR
ccactccctctctgcgcgctcgctcgctcactgaggccgggcgaccaaaggtcgcccgacgcccgggctttgcccgggcggcctcagtgagcgagcgagcgcgcagagaggga

SEQ ID NO: 6 - DNA, 190 bp, synthetic construct
  source 1..190: mol_type = other DNA; note = Spacer + JeT promoter + Kozak sequence
gcagaattcgccgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgttccggaaagccacc

SEQ ID NO: 7 - DNA, 57 bp, synthetic construct
  source 1..57: mol_type = other DNA; note = Spacer + PolyA Region
gtcgacgccaataaagagctcagatgcatcgatcagagtgtgttggttttttgtgtg

SEQ ID NO: 8 - DNA, 20 bp, synthetic construct
  source 1..20: mol_type = other DNA
cttgggtggagaggctattc

SEQ ID NO: 9 - DNA, 20 bp, synthetic construct
  source 1..20: mol_type = other DNA
aggtgagatgacaggagatc

SEQ ID NO: 10 - DNA, 22 bp, synthetic construct
  source 1..22: mol_type = other DNA
tacctactcactacccacgagc

SEQ ID NO: 11 - DNA, 20 bp, synthetic construct
  source 1..20: mol_type = other DNA
acacagaagaagaggcctgc

SEQ ID NO: 12 - DNA, 27 bp, synthetic construct
  source 1..27: mol_type = other DNA
gctctaccccattctacatctccagac

SEQ ID NO: 13 - DNA, 30 bp, synthetic construct
  source 1..30: mol_type = other DNA
gtgagttcaagagccatgttgctgatggtg

SEQ ID NO: 14 - DNA, 30 bp, synthetic construct
  source 1..30: mol_type = other DNA
tcgtgctttacggtatcgccgctcccgatt

SEQ ID NO: 15 - DNA, 20 bp, synthetic construct
  source 1..20: mol_type = other DNA
cctactcactgcccaagagc

SEQ ID NO: 16 - DNA, 20 bp, synthetic construct
  source 1..20: mol_type = other DNA
ctatgcggtagttcccggtg

SEQ ID NO: 17 - DNA, 20 bp, synthetic construct
  source 1..20: mol_type = other DNA
ggatatgctgatccccaccg

SEQ ID NO: 18 - DNA, 20 bp, synthetic construct
  source 1..20: mol_type = other DNA
acgaactcgctcttaggcag

(19) United States
(12) Patent Application Publication
(10) Pub. No.: US 2026/0297618 A1
(43) Pub. Date: Oct. 1, 2026

(54) GM2 ACTIVATOR VECTORS AND METHODS FOR USE THEREOF

(71) Applicant: GENCORREX THERAPIES INC., Kingston (CA)

(72) Inventor: Jagdeep WALIA, Kingston (CA)

(21) Appl. No.: 19/478,972

(22) PCT Filed: Apr. 26, 2024
(86) PCT No.: PCT/CA2024/050557

§ 371 Date: Oct. 27, 2025

Related U.S. Application Data
(60) Provisional application No. 63/462,660, filed on Apr. 28, 2023.

(51) Int. Cl. C12N 15/86; A61K 9/51; C07K 14/47; C12N 15/88

(52) U.S. Cl. CPC C12N 15/86; A61K 9/5123; C07K 14/4748; C12N 15/88; C12N 2750/14143; C12N 2830/008

(57) ABSTRACT

This disclosure relates to nucleic acid constructs for expression of GM2 activator (GM2A) protein, and viral vectors comprising said constructs useful for the treatment of AB-variant GM2 Gangliosidosis (ABGM2). Also provided are methods and uses of the vectors disclosed herein for the treatment of ABGM2.

### RELATED APPLICATION

This disclosure claims benefit of U.S. Provisional Patent Application Ser. No. 63/462,660 filed Apr. 28, 2023, incorporated herein by reference in its entirety.

### INCORPORATION OF SEQUENCE LISTING

A computer readable form of the Sequence Listing “29669-P70587PC00_SequenceListing.xml” (16,173 bytes), created on Apr. 22, 2024, is herein incorporated by reference.

### FIELD

The present disclosure relates to the development of a nucleic acid construct comprising a transgene encoding the DNA sequence for a GM2 activator (GM2A) protein operably linked to a promoter, and a transcription termination site; vectors comprising said nucleic acid constructs; pharmaceutical compositions comprising said vector; and vectors or compositions for use in the treatment of AB-variant GM2 Gangliosidosis (ABGM2).

### INTRODUCTION

AB-Variant GM2 Gangliosidosis (ABGM2) is a rare genetic disorder that results in progressive, widespread neuronal apoptosis and premature patient death. The disease manifests through a mutation in the GM2A gene, which encodes for the GM2 activator protein (GM2AP), an essential transport protein in the degradation of GM2 ganglioside. Disrupting the cell's natural ability to degrade GM2 ganglioside results in toxic accumulation that presents with developmental regression and impaired motor skills and death by age four, in humans¹. At present, there are limited treatment options and no cure².

Gene therapy is a plausible curative solution for GM2 Gangliosidosis³⁻⁵. In theory, introducing functional GM2A into a cell would result in their ability to produce an otherwise mutated protein and restore the cell's natural ability to degrade GM2 ganglioside. Viral vectors encapsulating the transgene of interest are widely used in the field, as they are safe and effective in transporting foreign genetic material⁶. Of these, adeno-associated viral (AAV) vectors have been extensively investigated in the context of gene therapy for human central nervous system (CNS) disorders⁷. They demonstrate stable transgene expression in the human brain⁸, low immune responses compared to other vectors⁹, and low risk of insertional mutagenesis¹⁰. More specifically, AAV serotype 9 (AAV9) has the unique ability to cross the blood-brain-barrier^{11,12}, which is a limiting step in the transduction of neuronal cells, crucial in treatments for CNS disorders.

Viral genomes contain cis-acting elements that are critical in transgene expression and gene stability, including promoters. A commonly used promoter is the JeT promoter, which is a unique combination of transcription factor binding sites to promote transcriptional activity that is comparable to other strong mammalian promoters¹³. A previous study demonstrated that the JeT promoter was equally as beneficial as using a strong CMV promoter in the CNS^{14,15}.

Codon optimization, a recombinant DNA technique used to replace codons in a gene sequence with synonymous ones, is another factor that aims to increase the rate and efficiency of translation. The genetic code is somewhat redundant in that most amino acids are encoded by more than one codon; thus, protein expression could be increased by using codons that are more abundant in the organism of interest^{16,17}. There is evidence that suggests codon optimization improves protein production due to mRNA transcription, likely as a result of increased GC content¹⁸⁻²⁰. While codon optimization can be unpredictable, there have been studies demonstrating the more efficient translation of codon-optimized transgenes versus wild-type genes²¹.

### SUMMARY

The present disclosure relates to nucleic acid constructs encoding functional GM2A protein operably linked to a promoter and transcription termination site, as well as viral vectors comprising said nucleic acid constructs for therapeutic replacement of dysfunctional GM2A protein. This disclosure also relates to the production of AAV vectors including nucleic acids encoding the GM2A protein.

The present inventors have tested the first CNS-directed, AAV9-based gene therapy for the treatment of ABGM2. It was found that delivery of GM2A plasmid DNA to cellular models of GM2A-D effectively restored protein and mRNA expression of GM2A. In murine models of ABGM2, treatment with scAAV9.JeT.coGM2A delivered intrathecally, resulted in decreased GM2 ganglioside accumulation throughout the mid-section of the brain. Overall, it was found that scAAV9.JeT.coGM2A represents a promising gene therapy approach to treating ABGM2.

Accordingly, the present disclosure provides a nucleic acid construct comprising a promoter, a transcription termination site, and a nucleotide sequence encoding a GM2A protein. It further provides a viral vector comprising said nucleic acid construct and methods of treating and preventing ABGM2 in a subject.

One aspect of the disclosure includes a nucleic acid construct comprising a nucleotide sequence encoding a GM2A protein operably linked to a promoter and a transcription termination site.

In an embodiment, the nucleotide sequence encoding a GM2A protein has a sequence as set forth in SEQ ID NO: 1, or a functional variant thereof.

In an embodiment, the GM2A protein has an amino acid sequence that is at least 50%, at least 60%, at least 70%, at least 80%, at least 90%, at least 95%, or at least 99% identical to the protein encoded by SEQ ID NO: 1, and which retains GM2A activity.

In an embodiment, the nucleic acid construct encodes a GM2A protein comprising an amino acid sequence set forth in SEQ ID NO: 2, or a functional variant thereof.

An aspect includes a viral vector comprising a nucleic acid construct described herein.

In an embodiment, the viral vector is an AAV vector, optionally AAV1, AAV2, AAV5, AAV6, AAV7, AAV8, and AAV9, AAVrh10 or a derivative thereof.

An aspect includes a pharmaceutical composition comprising a nucleic acid construct or viral vector described herein and a pharmaceutically acceptable carrier or diluent for example, including but not limited to, liposomes and lipid nanoparticles.

An aspect includes a method of treating or preventing AB-variant GM2 gangliosidosis (ABGM2) in a subject in need thereof, comprising administering to the subject a therapeutically effective amount of a viral vector or pharmaceutical composition described herein.

Another aspect includes a use of the viral vector or pharmaceutical composition described herein for the treatment or prevention of ABGM2.

A further aspect includes a use of the viral vector or pharmaceutical composition described herein for the manufacture of a medicament for the treatment or prevention of ABGM2. Also provided is the viral vector or pharmaceutical composition described herein for use in the treatment or prevention of ABGM2.

Another aspect includes a kit comprising the nucleic acid construct, the viral vector, or the pharmaceutical composition described herein and instructions for use.

These and other features and advantages of the present disclosure will become apparent from the following detailed description taken together with the accompanying drawings. It should be understood, however, that the detailed description and specific examples, while indicating preferred implementations of the present disclosure, are given by way of illustration only, since various changes and modifications within the spirit and scope of the disclosure will become apparent to those of skill in the art from this detailed description.

### DRAWINGS

Further objects, features and advantages of the disclosure will become apparent from the following detailed description taken in conjunction with the accompanying figures showing illustrative embodiments of the disclosure, in which:

FIG. 1 is a set up for intrathecal injections. The mouse is anesthetized in an induction chamber prior to being placed on the nose cone. A 15-ml conical tube is placed under the hips of the mouse with its nose secured in the nose cone.

FIG. 2 is the sectioning of CNS tissues during euthanizations of mice. The tissues of the CNS are divided into three brain sections and two spinal cord sections to analyze the distribution of vector. Sections were further divided for all analyses. Tissues for qPCR analysis of the brain were collected and mixed together and split into two sections one for RNA isolation and one for DNA isolation to perform gene expression. Other sections were collected for biochemical analysis by LC-MS/MS, western blotting and histology analysis.

FIG. 3 is the scAAV9.JeT.coGM2A construct design. Inverted terminal repeats (ITRs) flank the promoter, transgene, and polyadenylation sequence (polyA). One of the ITRs is mutated (A) to create self-complementary AAV. The vectors contain either a CBh, a CBh (m) or JeT promoter, driving expression of a wild-type (WT) or codon optimized (co) GM2A. Vector 1 contains CBh and WT GM2A. Vector 2 contains CBh (m) and coGM2A. Vector 3 contains JeT and coGM2A.

FIG. 4 is the GM2A gene expression in two different cell lines. All plasmids were transfected into two different GM2A knockout cell lines, MDA-MB-231 and HEK293, to determine gene expression by mRNA quantification. mRNA transcripts were detected in both cell lines as a result of transfection with each plasmid. Transcripts were measured as copies per uL. Data are expressed as mean±SEM. FIG. 4A shows MDA-MB-231 GM2A knockout cells transfected with CBh.wtGM2A produced significantly more transcripts compared to a GFP control (p<0.01 [*]; 1-way ANOVA; n=3 wells per transfection). All other cohort differences are insignificant (1-way ANOVA; n=3 wells per transfection). FIG. 4B shows that there were no significant differences between any of the cohorts following plasmid transfection in HEK293 GM2A knockout cells (1-way ANOVA; n=3 wells per transfection). GFP was used as a negative control.

FIG. 5 is the relative GM2A protein expression in MDA-MB-231 GM2A knockout cells. All plasmids were transfected into MDA-MB-231 GM2A knockout cells.

FIG. 5A shows a western blot of cell lysates collected and analyzed for GM2A protein expression. The bands migrating at ~20 kDa depict the mature protein, and the band migrating at ~22 kDa depicts the precursor protein for GM2A²⁵. β-actin (42 kDa) was used as the internal control. FIG. 5B shows quantification of GM2A protein expression in western blots. The band intensities of the mature and precursor forms of GM2A were taken together to represent total GM2A signal. Densitometry was conducted to quantify band intensities and each sample was normalized to β-actin intensity (n=4/cohort). Data are expressed as mean±SEM. The expression level of GM2A from all three plasmids were significantly greater than both controls (WT and GFP, p<0.0001 [****]; 1-way ANOVA; n=4/cohort; significance bars not shown). JeT.coGM2A also produced significantly higher levels of GM2A protein than both CBh.wtGM2A and CBh (m).coGM2A (p<0.0001 [****]; 1-way ANOVA; n=4/cohort). FIG. 5C shows GM2A protein expression relative to WT controls. JeT.coGM2A produced significantly higher levels of GM2A protein, relative to WT expression, than both CBh.wtGM2A and CBh (m).coGM2A (p<0.0001 [****]; 1-way ANOVA; n=4/cohort). WT: wild-type. GFP: green fluorescence protein.

FIG. 6 is the relative GM2A protein expression in HEK293 GM2A knockout cells. All plasmids were transfected into HEK293-GM2A knockout cells. FIG. 6A shows a western blot of cell lysates collected and analyzed for GM2A protein. The bands migrating at ~20 kDa depict the mature protein, and the band migrating at ~22 kDa depicts the precursor protein for GM2A²⁵. β-actin (42 kDa) was used as the internal control. FIG. 6B shows quantification of GM2A protein expression in western blots. The band intensities of the mature and precursor forms of GM2A were taken together to represent total GM2A signal. Densitometry was conducted to quantify band intensities and each sample was normalized to β-actin intensity (n=4/cohort). Data are expressed as mean±SEM. The expression level of GM2A from all three plasmids were significantly greater than both controls (WT and GFP, p<0.0001 [****]; 1-way ANOVA; n=4/cohort; significance bars not shown). FIG. 6C shows GM2A protein expression relative to WT controls. GM2A protein expression, relative to WT controls, was not significantly different between cells transduced with each of the three plasmids (1-way ANOVA; n=4/cohort). WT: wild-type. GFP: green fluorescence protein.

FIG. 7 is the GM2A protein expression per mRNA transcript, relative to WT control. The graph depicts the mean efficacy of the wild-type and codon optimized GM2A transgene by examining the ratio of GM2A protein expression (relative to WT control—FIG. 5C/6C) per mRNA transcript (FIG. 4A/B).

FIG. 8 is the relative GM2A protein expression in Lec2 cells. All vectors were transduced into Lec2 cells. FIG. 8A shows a western blot of cell lysates collected and analyzed for GM2A protein. The bands migrating at ~20 kDa depict the mature protein, and the band migrating at ~22 kDa depicts the precursor protein for GM2A²⁵. β-actin (42 kDa) was used as the internal control. FIG. 8B shows quantification of GM2A protein expression in western blots. The band intensities of the mature and precursor forms of GM2A were taken together to represent total GM2A signal. Densitometry was conducted to quantify band intensities and each sample was normalized to β-actin intensity (n=4/cohort). Data are expressed as mean±SEM. The expression level of GM2A from scAAV9.CBh.wtGM2A and scAAV9.JeT.coGM2A were significantly greater than the negative controls (untransduced Lec2 cells; p<0.0001 [****] and p<0.0032 [**], respectively; 1-way ANOVA; n=3/cohort; significance bars not shown). scAAV9.CBh.wtGM2A also produced more GM2A protein than scAAV9.JeT.coGM2A (p<0.0002 [***]; 1-way ANOVA; n=3/cohort). FIG. 8C shows GM2A protein expression relative to WT controls. GM2A protein expression, relative to WT controls, was significantly greater in scAAV9.CBh.wtGM2A-transduced cells versus scAAV9.JeT.coGM2A-transduced cells (p<0.0002 [***]; 1-way ANOVA; n=3/cohort)

FIG. 9 demonstrates that vectors efficiently biodistributed to the CNS and liver in vivo. ABGM2 mice were intrathecally injected with either scAAV9.CBh.wtGM2A, scAAV9.JeT.coGM2A or a vehicle (n=3/cohort). LSC, lumbar-section of the spinal cord; CSC, cervical-section of the spinal cord; CB, caudal-section of the brain; MB, mid-section of the brain. RB, rostral-section of the brain. Data are expressed as mean±SEM. FIG. 9A shows copy analysis conducted by ddPCR to determine the amount of transgene present in the CNS and liver. Data is presented as copies of GM2A (human GM2A) per diploid mouse genome (murine LaminB2). LaminB2 was used as the internal control. Transgene copy number was consistent in the CNS, regardless of what vector was injected; however, in the liver, the scAAV9.CBh.wtGM2A vector-injected cohort had significantly higher transgene present than the scAAV9.JeT.coGM2A vector-injected cohort (p<0.0001 [****]; 1-way ANOVA; n=3/cohort). FIG. 9B shows GM2A gene expression by RNA quantification conducted by ddPCR in the caudal- and mid-sections of the brain. Data is presented as copies of GM2A per uL. In both sections, the scAAV9.CBh.wtGM2A vector-injected cohort had significantly higher GM2A mRNA transcripts than the scAAV9.JeT.coGM2A vector-injected cohort (CB: p<0.0001 [****]; MB: p<0.0005 [***]; 1-way ANOVA; n=3/cohort).

FIG. 10 demonstrates that AAV-mediated gene therapy reduces GM2 accumulation in ABGM2 murine brains. Analysis of GM2 in the mid-section of murine brains was analyzed. GM2 levels are expressed as a function of GD1a, an internal control. Cohorts injected with either scAAV9.CBh.wtGM2A or scAAV9.JeT.coGM2A appear to have a reduced accumulation of GM2, compared to ABGM2 mice injected with a vehicle. GM2 levels are expressed as a function of GD1a, an internal control, which is a ubiquitous ganglioside highly expressed in brain tissue. These differences were not significant (1-way ANOVA; n=3/cohort).

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### I. General Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

All numerical values herein are modified by “about” or “approximately” the indicated value, and take into account experimental error and variations that would be expected by a person having ordinary skill in the art.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used herein, the singular forms “a”, “an”, and “the” include plural references unless the context clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to inclusive or be open-ended, i.e., to mean including but not limited to, and do not exclude additional, unrecited elements or process steps. Only the transitional phrases “consisting of” and “consisting essentially of” shall be closed or semi-closed transitional phrases, respectively.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

As used herein, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

### II. Nucleic Acid Constructs and Viral Vectors

Described herein is a nucleic acid construct comprising a nucleotide sequence encoding a GM2A protein, operably linked to a promoter and a transcription termination site. As shown herein, delivery of such nucleic acid constructs via adeno-associated viral vectors results in increased GM2A expression and decreased GM2 ganglioside accumulation in mouse models of ABGM2. Accordingly, one aspect of the disclosure includes a nucleic acid construct comprising a nucleotide sequence encoding a GM2A protein operably linked to a promoter and a transcription termination site.

The term “nucleic acid construct of the disclosure” as used herein refers to a nucleic acid molecule comprising an expression cassette, the expression cassette comprising a DNA sequence encoding a GM2A protein operably linked to a promoter and a transcription termination site. In an embodiment, the DNA sequence encoding a GM2A protein comprises a known GM2A nucleotide sequence. In an embodiment, the DNA sequence encoding a GM2A protein comprises a nucleotide sequence set forth in SEQ ID NO: 1 or a functional variant thereof. In an embodiment, the nucleic acid encodes a GM2A protein having an amino acid sequence as set forth in SEQ ID NO: 2 or a functional variant thereof.

The term “GM2 activator” protein or “GM2A” or “GM2AP” as used herein refers to a transport protein which participates in the degradation of the ganglioside GM2, and other molecules containing N-acetyl hexosamines. Defects in this gene have been implicated in GM2-gangliosidosis type AB or the AB variant of Tay-Sachs disease. Transcript variants due to alternative splicing have been described for this gene. For example, the nucleotide and amino acid sequence of human GM2A can be found for at GenBank ID: 2760 and UniProt ID: P17900, including all isoforms.

The term “GM2A activity” as used herein refers to a protein that is known to act as a substrate specific co-factor to catalyze the degradation of GM2 ganglioside.

The term “nucleic acid molecule” and its derivatives, as used herein, are intended to include unmodified DNA or RNA or modified DNA or RNA. For example, the nucleic acid molecules or polynucleotides of the disclosure can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “operably linked” as used herein refers to a relationship between two components that allows them to function in an intended manner. For example, where a coding sequence is operably linked to a promoter, the promoter actuates expression of the coding sequence.

The term “promoter” or “promoter sequence” generally refers to a regulatory DNA sequence capable of being bound by an RNA polymerase to initiate transcription of a downstream (i.e. 3′) sequence to generate an RNA. Suitable promoters may be derived from any organism and may be bound or recognized by any RNA polymerase. Suitable promoters for the expression cassette will be known to the skilled person. In some embodiments, the promoter is an inducible promoter. Examples of inducible promoters include, without limitation, a tetracycline response element (TRE) (e.g. Tet-ON or Tet-OFF systems), ponA-inducible expression systems (Agilent Technologies), or cumate-inducible promoters such as CuO (System Biosciences). In some embodiments, the promoter is a constitutive promoter. Examples of constitutive promoters include human Ubiquitin C (UBC), human Elongation Factor 1a (EF1A), human phosphoglycerate kinase 1 (PGK), simian virus 40 early promoter (SV40) (GeneBank accession number J02400.1), cytomegalovirus immediate-early promoter (CMV), chicken b-Actin promoter coupled with CMV early enhancer (CAG), chicken β-actin hybrid (CBh) and EF1-HTLV. In some embodiments, the promoter is a tissue- or cell-specific promoter. In an embodiment, the promoter is a synthetic promoter such as JeT.

The term “transcription termination site” as used herein refers generally to a polyadenylation signal (pA) that terminates transcription of messenger RNA (mRNA). As used herein, the phrase “polyadenylation signal” refers to sequences from various genes that can be added to mammalian vectors to ensure proper mRNA processing and stability. For example, a 100-200 nucleotide polyadenylate tail can be added to the 3′ end of a coding sequence to protect mRNA from degradatory action of phosphatases and nucleases. Suitable pAs may be derived from any organism and are known to the skilled person. Examples of pA signals include, without limitation, rabbit beta-globin pA (GeneBank accession number K03256), SV40 late polyA, and hGH polyA and strong bovine growth hormone pA (BGHpA).

The term “functional variant” as used herein includes modifications of the nucleic acid or polypeptide sequences disclosed herein that perform substantially the same function as the nucleic acid molecules or polypeptides disclosed herein in substantially the same way. For example, the functional variant may comprise sequences having at least 50%, or at least 60%, or at least 70%, or at least 80%, or at least 90%, or at least 95%, or at least 99% sequence identity to the sequences disclosed herein. In the case of nucleic acids, functional variants include nucleotide sequences that hybridize to the nucleic acid sequences set out above, under at least moderately stringent hybridization conditions, optionally stringent hybridization conditions, or the functional variant nucleic acid sequences may comprise degenerate codon substitutions or codon-optimized nucleic acid sequences. In the case of polypeptides, the functional variant may also comprise conservatively substituted amino acid sequences of the sequences disclosed herein.

In an embodiment, the functional variant sequences comprise sequences having at least 50%, or at least 60%, or at least 70%, or at least 80%, or at least 90%, or at least 95%, or at least 99% sequence identity to the sequences disclosed herein.

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=[number of identical overlapping positions]/[total number of positions]×100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In one embodiment, the functional variants include nucleotide sequences that hybridize to the nucleic acid sequences described herein, under at least moderately stringent hybridization conditions, optionally stringent hybridization conditions.

With reference to nucleic acids, the terms “anneal” and “hybridize” as used herein refer to the ability of a nucleic acid to non-covalently interact with another nucleic acid through base-pairing. The terms “complementary” or “complementary nucleic acid” refer to a nucleic acid or a portion of a nucleic acid that is able to anneal with a nucleic acid of a given sequence. In some cases, this is referred to as the “reverse complement” of a given sequence.

By “at least moderately stringent hybridization conditions” it is meant that conditions are selected which promote selective hybridization between two complementary nucleic acid molecules in solution. The term “at least moderately stringent hybridization conditions” encompasses stringent hybridization conditions and moderately stringent hybridization conditions. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41 (% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In some embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm−5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: a Laboratory Manual, Cold Spring Harbor Laboratory Press, 2001.

In another embodiment, the functional variant nucleic acid sequences comprise degenerate codon substitutions or codon-optimized nucleic acid sequences. The term “degenerate codon substitution” as used herein refers to variant nucleic acid sequences in which the second and/or third base of a codon is substituted with a different base that does not result in a change in the amino acid sequence encoded therein. The term “codon-optimized” as used herein refers to a variant nucleic acid molecule comprising one or more degenerate codon substitutions that reflect the codon usage bias of a particular organism. Accordingly, in an embodiment, the nucleic acid construct disclosed herein comprises a codon-optimized or degenerate nucleotide sequence of SEQ ID NO:1.

In an embodiment, the nucleic acid construct disclosed herein comprises a nucleic acid molecule that encodes a polypeptide having an amino acid sequence that is at least 50%, at least 60%, at least 70%, at least 80%, at least 90%, at least 95%, or at least 99% identical to the protein encoded by SEQ ID NO:1, and which retains GM2A activity.

In an embodiment, the nucleic acid construct further comprises, an enhancer, a post-transcription regulatory sequence, one or more sequences that facilitate incorporation of the nucleic acid into a viral particle and/or integration into the host genome, or any combination thereof, operably linked to the nucleic acid encoding the GM2A protein. Post transcriptional regulatory sequences include, for example, without limitation, sequences of nucleotides that when placed in an AAV transfer plasmid results in the increased or decreased expression of the transgene. As used herein, the phrase “enhancer” refers to a sequence of nucleotides that argument the activity of a promoter in an orientation, position, and distance-dependent manner. Enhancers play a significant role in the regulation of tissue-specific gene expression in high eukaryotes but have been repurposed for use in recombinant DNA technologies to impact the transcriptional activity of an associated promoter. Typically, a trans-acting gene regulatory protein binds the enhancer in order to affect transcriptional activity of the associated promoter.

In some embodiments, the nucleic acid construct comprises a sequence set out in SEQ ID NO: 3, or a functional variant thereof.

### Viral Vectors

Also described herein is a viral construct comprising a nucleic acid construct described herein. Viral constructs are made of DNA or RNA and they contain some of the genetic material of the viruses they are derived from (such as lentivirus, retrovirus, AAV and adenoviruses). For example, viral constructs may include sequences that facilitate incorporation of the nucleic acid into a viral particle and/or integration into the host genome. In some embodiments, the viral construct may include inverted terminal repeats (ITRs) for example from an AAV such as AAV9, or other viral sequences. Viral constructs have been modified to carry and to deliver a gene of interest that will produce a protein or an RNA of interest and can be used for example for the treatment of diseases by gene therapy. Suitable viral constructs are known in the art and depend on the type of viral vectors and viruses being used.

One aspect of the disclosure is a viral vector comprising a nucleic acid construct disclosed herein. Replication incompetent viral vectors are particularly useful in gene therapy applications as they allow for efficient transduction of delivery of a transgene to target tissues. Differences between viral vectors include availability of tropisms, packaging capacity, safety, and transduction efficiencies in different tissues.

The term “viral vector” as used herein is intended to include viral particles or virus-like particles capable of transduction of a target cell. Common viral vectors include, but are not limited to, HIV-derived lentiviral vectors, retroviral vectors, adenoviral vectors, and recombinant adeno-associated virus (AAV) vectors. Other viral vectors may be derived from rhabdovirus (such as vesicular stomatitis virus (VSV)), or herpes virus (such CMV and HSV-1). Typical components of the viral vector are the structural components of the viral particle, such as the proteins making the capsid and the envelope of the vector. Other components are the enzymes involved in the replication of the vector RNA or DNA. Such enzymes can be also involved in the synthesis, maturation or transport of the virus RNA. These enzymes can also be involved in the processing and maturation of viral components, as well as in the integration of the genome of the virus into the cell chromosomes. Enzymes that are components of the viral vectors can also be involved in the reverse transcription of the virus genomic RNA into DNA. Other components of the vector can be protein or peptide that regulate the replication, transcription, transport or translation of the genes or gene products of the viral vector. Such factors can also activate or decrease the expression of cellular genes and they can modulate the defense mechanism of the cells against viruses.

Several viral vectors are well known in the art including adenovirus, adenoviral associated virus (AAV), lentivirus, retrovirus, and herpes simplex virus 1. Accordingly, in an embodiment, the viral vector is a lentivirus, adenovirus, adenoviral associated virus (AAV), retrovirus, or herpes simplex virus 1 vector. Optionally, the viral vector is an AAV vector.

AAV is particularly useful for gene therapy applications as it elicits a limited immune response, exhibits a wide variety of serotypes, and has a stable expression profile. Accordingly, in an embodiment, the viral vector is an AAV vector or a derivative thereof. Optionally, the AAV vector is selected from the group consisting of AAV1, AAV2, AAV5, AAV6, AAV7, AAV8, AAV9, AAVr10 and derivatives thereof.

The term “AAV derivative” as used herein describes a recombinant AAV produced by combining AAV helper plasmids from different AAV serotypes to produce AAV capsids with the combined advantages of more than one serotype. An AAV derivative may further refer to a shuffled AAV derivative which used herein describes an AAV virus containing mutations produced through directed evolutionary or related recombination techniques including but not limited to DNA shuffling. The term “AAV derivative” may also refer to a capsid-modified AAV that can be produced by pseudo typing the sequences of two or more AAV serotypes producing an AAV vector combining characteristics of the two or more serotypes. In an embodiment, the AAV vector is a chimeric, shuffled or capsid modified derivative of AAV.

### Compositions and Kits

In one embodiment there is provided a pharmaceutical composition comprising a nucleic acid construct or viral vector described herein or a derivative of it, and a pharmaceutically acceptable carrier or diluent. The composition may be formulated for use or prepared for administration to a subject using pharmaceutically acceptable formulations known in the art including liposomes or lipid nanoparticles. Conventional procedures and ingredients for the selection and preparation of suitable formulations are described, for example, in Remington's Pharmaceutical Sciences (2003—20^{th} edition) and in The United States Pharmacopeia: The National Formulary (USP 24 NF19) published in 1999. The term “pharmaceutically acceptable” means compatible with the treatment of animals, in particular, humans.

On this basis, the pharmaceutical compositions could include an active compound or substance, such as a nucleic acid construct or viral vector described herein, in association with one or more pharmaceutically acceptable vehicles or diluents, and contained in buffered solutions with a suitable pH and isosmotic with the physiological fluids. The methods of combining viral vectors the vehicles or combining them with diluents is well known to those skilled in the art. The composition could include a targeting agent for the delivery or transport of the active compound to specified sites within the body, organ, tissue, or cell.

As used herein, the term “diluent” refers to a pharmaceutically acceptable carrier which does not inhibit a physiological activity or property of an active compound, such as lipoxin or a lipoxin analogue, to be administered and does not irritate the subject and does not abrogate the biological activity and properties of the administered compound. Diluents include any and all solvents, dispersion media, coatings, surfactants, antioxidants, preservative salts, preservatives, binders, excipients, disintegration agents, lubricants, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical compositions, formulations, dosages, etc. described herein can be administered for example, by parenteral, intravenous, intrathecal, subcutaneous, or intramuscular administration in dosage unit formulations containing conventional non-toxic pharmaceutically acceptable carriers, adjuvants and vehicles.

The nucleic acid constructs or viral vectors described herein are suitably formulated in a conventional manner into compositions using one or more carriers or diluents. Accordingly, the present description also includes a composition comprising one or more nucleic acid constructs or viral vectors described herein and a carrier or diluent. The nucleic acid constructs or viral vectors described herein are suitably formulated into pharmaceutical compositions for administration to subjects in a biologically compatible form suitable for administration in vivo. Accordingly, the present description further includes a pharmaceutical composition comprising the nucleic acid constructs or viral vectors described herein, and a pharmaceutically acceptable carrier. In some embodiments the pharmaceutical compositions are used in the treatment of any of the diseases, disorders or conditions described herein. In an embodiment, the disease, disorder, or condition is ABGM2.

In some embodiments, the nucleic acid constructs or viral vectors described herein are formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, nucleic acid constructs or viral vectors described herein are suitably in a sterile powder form for reconstitution with a suitable vehicle, e.g., sterile pyrogen-free water, before use.

Also provided are kits comprising a nucleic acid construct, viral vector, or pharmaceutical composition as described herein, along with suitable container or packaging and/or instructions for the use thereof, such as for the treatment of ABGM2 in a subject.

### III. Methods and Uses

As described in the Examples, scAAV9.JeT.coGM2A can effectively restore the protein and mRNA expression of GM2A while reducing GM2 ganglioside accumulation in treated cellular and murine models of AB-variant GM2 gangliosidosis (ABGM2). Accordingly, one aspect of the disclosure is a method of treating or preventing ABGM2 in a subject in need thereof, comprising administering a therapeutically effective amount of the pharmaceutical composition, the nucleic acid construct or the viral vector disclosed herein to the subject. Another aspect of the disclosure includes use of the pharmaceutical composition, the nucleic acid construct or the viral vector described herein to treat or prevent ABGM2. An aspect also includes use of the pharmaceutical composition, the nucleic acid construct or the viral vector described herein in the manufacture of a medicament for treating or preventing ABGM2. An aspect also includes the pharmaceutical composition, the nucleic acid construct or the viral vector described herein for use in treating or preventing ABGM2.

In an embodiment, the use for or method of treating or preventing ABGM2 comprises formulating for or administering the therapeutically effective amount of nucleic acid construct, the vector or the pharmaceutical composition disclosed herein by intravenous and/or intrathecal injection.

The term AB-variant GM2 Gangliosidosis (ABGM2) describes a rare genetic disorder inherited in an autosomal recessive manner. ABGM2 is caused by mutations in the GM2A gene that is characterized by developmental regression, impaired motor skills, accumulation of GM2 ganglioside in the brain, progressive, widespread neuronal apoptosis and premature death.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a patient in remission), preventing disease or preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. Treatment methods and uses comprise administering to a subject a therapeutically effective amount of the pharmaceutical composition, the nucleic acid construct or the viral vector described herein and optionally consists of a single administration or use, or alternatively comprises a series of administrations or uses.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a subject becoming afflicted with a disease, disorder or condition or manifesting a symptom associated with a disease, disorder or condition.

The term “subject” as used herein includes all members of the animal kingdom including mammals, and suitably refers to humans. Optionally, the term “subject” includes mammals that have been diagnosed with ABGM2. In an embodiment, the subject is a mammal. In another embodiment, the subject is human. In one embodiment, the term “subject” refers to a human having, or suspected of having, ABGM2.

The term “subject in need thereof” refers to a subject that could benefit from the method(s) or treatment(s) described herein, and optionally refers to a subject with ABGM2, or optionally a subject with increased risk of ABGM2, such as a subject with a strong genetic predisposition.

The term “administered” or “administering” as used herein means administration of a therapeutically effective amount of a compound or composition of the disclosure to a cell either in cell culture or in a subject. The nucleic acid constructs or viral vectors described herein may be administered to a subject in a variety of forms depending on the selected route of administration, as will be understood by those skilled in the art. For example, the nucleic acid constructs or viral vectors described herein may be administered by parenteral administration or direct injection into brain and the pharmaceutical compositions formulated accordingly. In some embodiments, administration is by means of a pump for periodic or continuous delivery.

The nucleic acid constructs or viral vectors described herein may be administered to or used in a subject in a variety of forms depending on the selected route of administration or use, as will be understood by those skilled in the art. For example, the nucleic acid constructs or viral vectors described herein may be administered by parenteral administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000—20^{th} edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, intrapulmonary (for example, by use of an aerosol), and intrathecal modes of administration. Parenteral administration may be by continuous infusion over a selected period of time.

As used herein, the phrase “intrathecal” means existing or taking place within, or administered into the fluid-filled space between the thin layers of tissue that cover the brain and spinal cord.

As used herein, the phrase “intravenous” means existing or taking place within, or administered into, a vein or veins. Intravenous delivery of gene therapy vectors allows for widespread delivery and transduction to organs and tissues in a subject.

As used herein, the phrase “effective amount” or “therapeutically effective amount” means an amount effective, at dosages and for periods of time necessary to achieve the desired result. For example, in the context of treating ABGM2, an effective amount is an amount that for example decreases the accumulation of GM2 ganglioside in the brain compared to the response obtained without administration of the compound. Effective amounts may vary according to factors such as the disease state, age, sex, and weight of the animal. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

Suitable use or administration schedules may include, without limitation, at least once a week, from about once in lifetime, one time per two weeks, three weeks or one month, about one time per week to about once daily. The length of the treatment period may depend on a variety of factors, such as the severity of the disease, disorder or condition, the age of the subject, the concentration and/or the activity of the nucleic acid constructs or viral vectors described herein. It will also be appreciated that the effective dosage of the nucleic acid constructs or viral vectors described herein used for the treatment may increase or decrease over the course of a particular treatment regime. Changes in dosage may result and become apparent by standard diagnostic assays known in the art. In some instances, chronic administration or use is required. For example, the nucleic acid construct or viral vector described herein are administered to or for use in the subject in an amount and for duration sufficient to treat the subject.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

### EXAMPLES

The following non-limiting examples are illustrative of the present disclosure.

### Example 1: Materials and Methods

### Cell Culture

MDA-MB, HEK293 and Lec2 cells were used in this study. The GM2A knockout MDA-MB and HEK293 cell lines were created in Walia lab. The GM2A gene was targeted by introducing a frameshift mutation in the first exon eliminating GM2A expression. Cells were maintained in DMEM and 10% FBS. Cells were maintained in an incubator at 37° C. and 5% CO₂.

### Plasmids and Vectors

The GM2A vectors include the human GM2A cDNA sequences. The nucleic acid construct is under the control of the synthetic JeT promoter and followed by a poly-adenylation signal. The entire sequence is flanked by inverted terminal repeats (ITR) to allow for packaging into the self-complimentary AAV9 (scAAV9) vector, with the 3′ ITR have a mutated terminal resolution site to allow for self-complimentary folding²². The designed vector was synthesized with a codon optimized transgene sequence of GM2A for optimal expression (Biobasics, Markham, ON). The plasmid was transformed by addition to competent E. coli bacterial cells and isolated by miniprep in accordance with the kit protocol (QIAprep Spin Miniprep Kit, Qiagen) for use in transfection. DNA concentration and purity were determined using a Nanodrop 2000 (Thermo Fisher). Plasmids were sent to Aldevron, LCC for larger plasmid prep and to UNC Vector Core for viral vector preparation (UNC Vector Core, UNC School of Medicine). To test the GM2A vector, GM2A knockout cells were transfected with the human GM2A vector. Protein isolate from transfected cells were also analyzed for GM2A protein expression.

### Transfections and Transductions

For transfection, cells were seeded in a 6-well plate at approximately 500,000 cells per well determined by manual cell counting using Trypan Blue (Gibco, 15250061). The GM2A plasmid or vector was introduced to the cells by Lipofectamine 3000 (Fisher Scientific, L3000001) mediated transfection according to the manufacturer's protocol. For transfection cells were treated with plasmids containing the human GM2A construct or a GFP plasmid to provide a visual of transfection efficiency. Untreated WT cells were used as a positive control and GM2A knockout cells treated with GFP plasmids were used as a negative control (Table 1). For transduction, cells were treated with the scAAV9.JeT.coGM2A vector. Wild-type Lec2 cells served as a control (Table 2). After 48 hours, cells were subject to protein or RNA isolation for their respective analyses.

TABLE 1
In vitro study design for transfection of cellular models of ABGM2.

| | | | Dose (μg of | |
|---|---|---|---|---|
| Cohort | Cell Type | Treatment | DNA) | Rationale |
| 1 | MDA-MB | Control | 0 | (+) Control |
| | GM2A +/+ | | | |
| 2 | HEK293GM2A | Control | 0 | (+) Control |
| | +/+ | | | |
| 3 | MDA-MB | Vehicle - GFP | 1.11 | (-) Control |
| | GM2A -/- | | | |
| 4 | HEK293GM2A | Vehicle - GFP | 1.11 | (-) Control |
| | -/- | | | |
| 5 | MDA-MB | GM2A | 0.66 | Cell Type 1 |
| | GM2A -/- | | | |
| 6 | HEK293 | GM2A | 0.66 | Cell Type 2 |
| | GM2A -/- | | | |

TABLE 2
In vitro study design for transduction of Lec2 cells.

| | Cell | | Dose | |
|---|---|---|---|---|
| Cohort | Type | Treatment | (MOI) | Rationale |
| 7 | Lec2 | None | 0 | Wild Type |
| 8 | Lec2 | SCAAV9.JeT.coGM2A | 10⁵ | Vector |

### Animal Models and In Vivo Study Design

A newly developed animal model for ABGM2 was used for this study. This model has both Gm2a and Neu3 knocked out and has been previously described and characterized in the Walia Lab (unpublished). These mice exhibit abnormal accumulation of GM2 Ganglioside, reduced life span as well as compromised behavioural parameters. These include reduced coordination and overall movement. These abnormalities are noted as early as 8 weeks of age.

The Neu3−/−Gm2a−/− double knockout mice were bred and monitored at Queen's University and maintained on 12-hr light cycle from 7 a.m. to 7 p.m. Colony upkeep and maintenance was done in the Animal Facility at Queen's University (Kingston, Ontario, Canada) and all procedures were performed in regulation with the University Animal Care Committee protocols.

For the in vivo study, animals were injected intrathecally with 6.5×10¹⁰ vector genomes (vg) per mouse of the scAAV9.JeT.coGM2A vector or a vehicle control (Table 3). Vectors were made up in 1×PBS with 5% sorbitol for the appropriate dosage while vehicle injections were performed with 1×PBS with 5% sorbitol only.

TABLE 3
Study design for in vivo study.

| | | Mice | Dose | Injection | Endpoint |
|---|---|---|---|---|---|
| Cohort | Genotype | (n) | (vg/mouse) | Type | (weeks) |
| 1 | Neu3-/-Gm2a^{-/-} | 3 | 0 | IT | 10 |
| 2 | Neu3-/-Gm2a^{-/-} | 3 | 6.5 × 10¹⁰ | IT | 10 |

### Genotyping

Genotyping was performed on DNA extracted from ear notches collected from the mice at or before 21 days of age. DNA digestion was carried out using Q5® High-Fidelity 2× Master Mix (M0491L; New England BioLabs Ltd.). Samples were then prepared for polymerase chain reactions (PCR). The following primers were used to detect Gm2a and Neu3:

Gm2a Mutation Forward
(SEQ ID NO: 8)
5′-CTTGGGTGGAGAGGCTATTC-3′;
Gm2a Mutation Reverse
(SEQ ID NO: 9)
5′-AGGTGAGATGACAGGAGATC-3′;
Gm2a WT Forward
(SEQ ID NO: 10)
5′-TACCTACTCACTACCCACGAGC-3′;
Gm2a WT Reverse
(SEQ ID NO: 11)
5′-ACACAGAAGAAGAGGCCTGC-3′;
Neu3 Forward
(SEQ ID NO: 12)
5′-GCTCTACCCCATTCTACATCTCCAGAC-3′;
Neu3 Reverse:
(SEQ ID NO: 13)
5′-GTGAGTTCAAGAGCCATGTTGCTGATGGTG-3′;
Neu3 Neomycin Cassette:
(SEQ ID NO: 14)
5′-TCGTGCTTTACGGTATCGCCGCTCCCGATT-3′.

### Intrathecal (IT) Injections

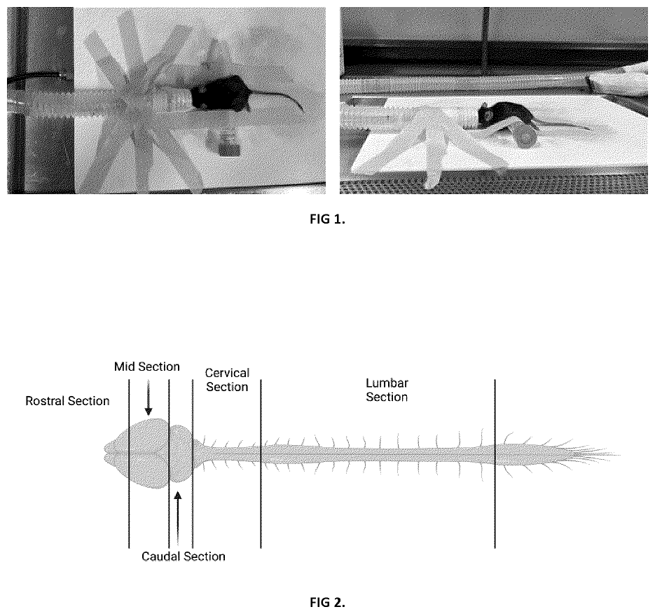

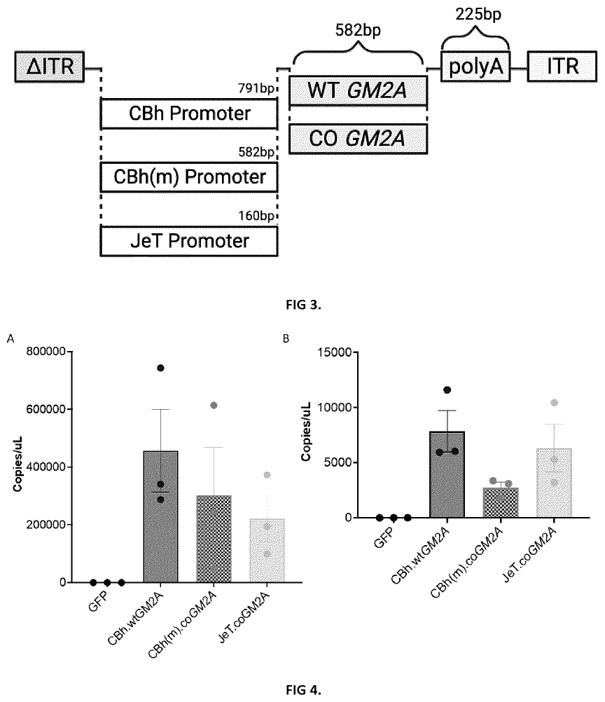

The protocol for performing intrathecal injections was adapted from previous literature^{23,24}. At 6 weeks of age, mice were anesthetized by inhalation of isoflurane. The mice were placed with their head in a nose cone while the hips are elevated by a 15-mL conical tube (FIG. 1). The back of the mouse was shaved and sterilized and the location between L5 and L6 was palpated to mark the injection spot. A Hamilton syringe with a 30-gauge needle was loaded with the vector at a volume of 15 μL for a dose of 6.5×10¹⁰ vg per mouse. The syringe was inserted at a 90° angle from the spine with the needle bevel facing up. Once it contacts the spinal column the syringe was bent to a 50-30° angle such that it can enter the subarachnoid space between L5 and L6. Proper penetration was indicated by a tail flick—a movement of the tail in the shape of an S. When in the appropriate spot, the vector was injected slowly, and the needle kept in place from a few seconds before turning the bevel down and being removed. The mice were recovered in a clean cage and monitored for several minutes after injections to ensure there is no paralysis caused by the injection.

### Immunosuppression and Blood Collection

To reduce the immune response against the vector, mice received an immunosuppression regimen, including rapamycin and prednisone, from 5 weeks of age up until their endpoint (10 weeks of age). Rapamycin and prednisone were both dissolved in dimethylsulfoxide (Thermo Fisher Scientific, Waltham, Massachusetts, United States) and diluted in 0.9% saline or phosphate buffered saline (PBS), respectively. Rapamycin (LC-Laboratories, R-5000) had a loading dose of 300 g, followed by a daily dose of 100 g per day until euthanization. Prednisone (Sigma Aldrich, P6254) was administered at a dose of 0.24 g per day until euthanization.

### Euthanizations

Tissue samples were collected at the designated short-term endpoint of 10 weeks. The mice were euthanized by CO₂ asphyxiation after which a cardiac puncture was performed. Mice were then perfused with 10 ml of 1×PBS. Visceral organs collected include the liver, heart, gonad, lung, spleen, kidney, and muscle and were sectioned for their respective analyses. The brain was sectioned into rostral, mid-section and caudal regions while the spinal cord was sectioned into lumbar and cervical sections (FIG. 2). Organ designated for RNA isolation were stored in RNALater™ Solution (Invitrogen) at −80° C. All organs were frozen at −20° C. until processing for their respective analyses.

Droplet Digital Polymerase Chain Reaction (ddPCR)

DNA extraction was performed using an extraction kit obtained from Geneaid Biotech Ltd. (Xizhi District, New Taipei City, Taiwan) following the manufacturer protocol. Droplet digital polymerase chain reaction (ddPCR) was performed to assess vector biodistribution. Each sample was mixed with 2× QX200™ ddPCR™ EvaGreen® Supermix (Bio-Rad Laboratories, Hercules, California, United States), respective primers and nuclease-free water. 20 μL of each reaction was loaded into a DG8™ Cartridge for QX200™/QX100™ Droplet Generator (Bio-Rad Laboratories, Hercules, California, United States), followed by 70 μL of QX200 Droplet Generation Oil for EvaGreen® (Bio-Rad Laboratories, Hercules, California, United States). The generated droplets were then loaded into a PCR plate and run on the T100 PCR Gradient Thermal Cycler (Bio-Rad Laboratories, Hercules, California, United States). Primers for the transgenes are as follows: Wild-type transgene: (forward) 5′-CCTACTCACTGCCCAAGAGC-3′ (SEQ ID NO: 15), (reverse) 5′-CTATGCGGTAGTTCCCGGTG-3′ (SEQ ID NO: 16). Codon optimized transgene: (forward) 5′-GGATATGCTGATCCCCACCG-3′ (SEQ ID NO: 17), (reverse) 5′-ACGAACTCGCTCTTAGGCAG-3′ (SEQ ID NO: 18).

Gene expression was analyzed by isolation of RNA using GeneJET RNA Purification Kit (Thermo Fisher Scientific, Waltham, Massachusetts, United States) and the accompanied protocol. cDNA was synthesized using the QuantiTect Reverse Transcription Kit (Qiagen, Hilden, Germany). ddPCR was then performed on caudal- and mid-sections of the murine brain to quantify gene expression, as described above.

### Western Blotting

To extract protein lysate from the cellular models, 1× radioimmunoprecipitation assay (RIPA) buffer (Cell Signalling Technology, 9806) was added to each well and the cells were briefly incubated on ice. The cells were scraped into tubes and sonicated (20% power, 10 seconds/sample, twice). Cell debris was removed by centrifugation. Tissue samples were weighed out and added to a bead tube for homogenization with RIPA buffer. The homogenate was incubated in the RIPA buffer for 10 minutes on ice. The tissue was sonicated (20% power, 10 seconds/sample, twice) and debris was removed by centrifugation and collection of the supernatant. Protein concentration was determined using the Pierce™ BCA Protein Assay Kit following the manufacturer protocol (Thermo Fisher, 23225).

Western blots were performed in accordance with most standard protocols. Briefly, 25 μg of protein was loaded into each well along with a ladder (Precision Plus Protein™ Kaleidoscope™ Prestained Protein Standards, 1610375, BioRad) and proteins were separated via SDS-PAGE on a 12% polyacrylamide gel. The proteins were transferred to a nitrocellulose membrane. The membrane was blocked with a 5% skim milk solution and then incubated overnight with the primary antibody against GM2A (Antibody Solutions, Santa Clara, CA 95054, USA). Following several washes, the secondary antibody Mouse anti-goat IgG-HRP (Santa Cruz Biotechnology, sc-2354, lot A1921) was added followed by another set of wash steps. Proteins were visualized by the chemiluminescent detection method using Immobilon Western chemiluminescent HRP substrate reagents (Millipore Sigma, WBKLS0500). The western blot was imaged using the Azure Biosystems C600 imaging system. The β-Actin protein was used as an internal control to show equal protein loading between wells. The membrane was washed following imaging for the GM2A target protein and then incubated with the primary β-actin antibody produced in rabbit (Sigma Aldrich, A2066-100UL) overnight and imaged following the same steps as above for secondary antibody staining Anti mouse IgG-HRP (Santa Cruz Biotechnology, sc-25409,).

### Statistical Analysis

All statistical analysis was performed in Graph Pad Prism 9. In most cases, a one-way ANOVA with a Tukey's multiple comparison test was used to compare treatment groups.

### Example 2: An In Vitro Study Showed Restored GM2A Expression in a Treated Cellular Model of GM2A Deficiency

To confirm that the construct could successfully restore GM2A expression when delivered to cells, in vitro experiments were performed with plasmid DNA. For all below experiments, untransfected WT cells or cells treated with a plasmid containing GFP only were used as controls. Cells were transfected using Lipofectamine™ 3000 according to the kit protocol.

GM2A Expression was Restored Following Transfections of Cellular Models of ABGM2 with Plasmids Carrying the Designed GM2A Construct Confirmed by ddPCR and WB.

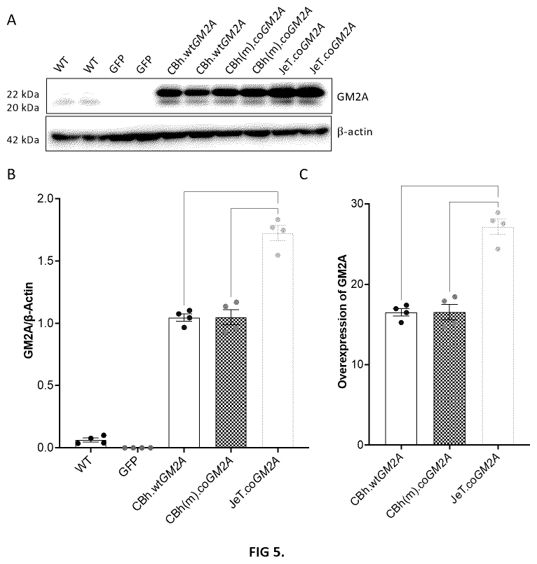

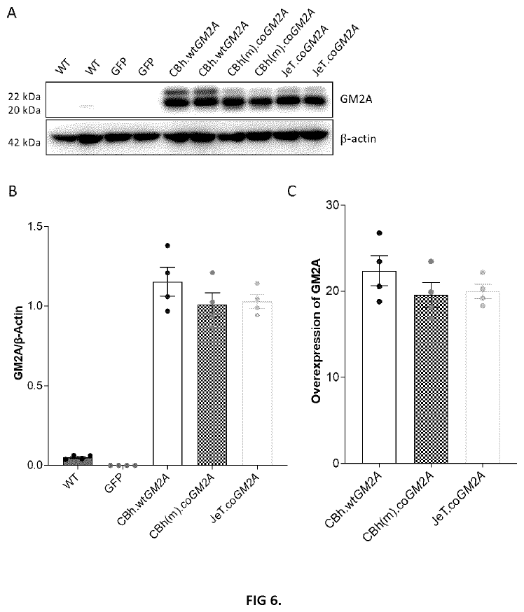

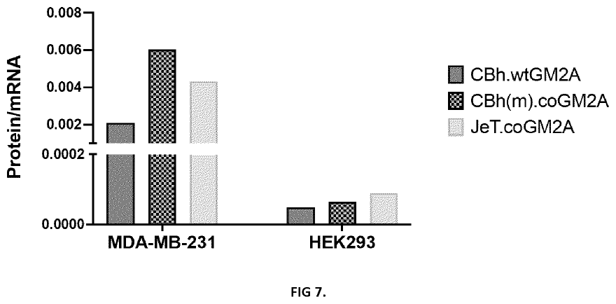

Following transfections with the GM2A plasmid, cells were lysed and subject to RNA isolation for ddPCR and protein extraction for western blots. For ddPCR, copy number data is measured in copies per uL. These experiments confirmed that the plasmids were successfully transfecting the cells and generating copies of GM2A mRNA (FIG. 4). To determine protein expression, a western blot was conducted and GM2A was measured relative to β-Actin, a reference gene in MDA-MB cells (FIG. 5A) and HEK293 cells (FIG. 6A). The expression of GM2A was significantly increased following treatment of knockout cells with JeT.coGM2A compared to both the GFP and WT controls in MDA-MB cells (FIG. 5B,C) and HEK293 cells (FIG. 6B,C). This data indicated that the GM2A plasmid can successfully restore gene expression in a cellular model of ABGM2.

### Example 3: An In Vitro Study Showed GM2A Vectors Efficacy in a Treating Cellular Model of GM2A Deficiency

To confirm that the vector could successfully restore GM2A expression when delivered to cells, in vitro experiments were performed with scAAV9.JeT.coGM2A. A cellular model of ABGM2 was created and was also confirmed by using a Lec2 cell line. For all below experiments, wildtype (WT) cells were used as negative controls. Cells were transduced using Lipofectamine™ 3000 according to the kit protocol.

GM2A Protein Expression was Restored Following Transduction of Lec2 Cells with Vectors Carrying the Designed GM2A Construct.

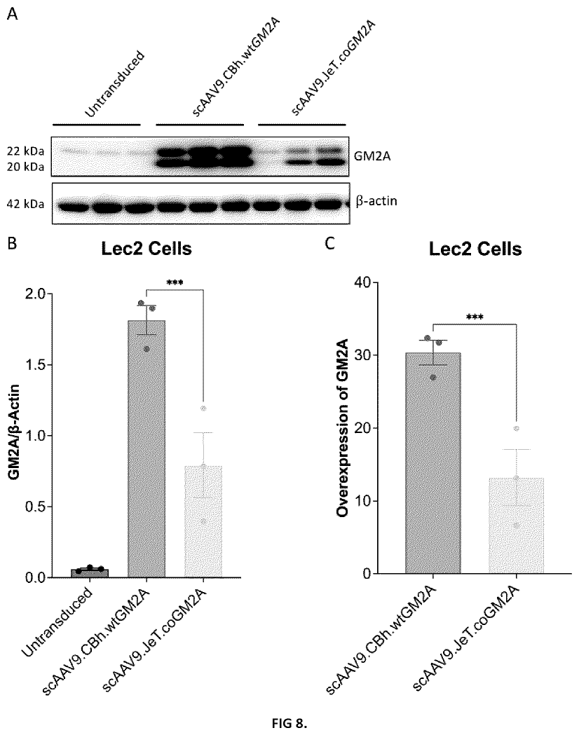

To confirm protein expression could be restored following transduction of Lec2 cells, a western blot was performed (FIG. 8A). Cells were transduced in a 6-well plate. An MOI of 105 was used. The β-Actin protein was used as an internal control. It was evident that transfection of GM2A KO cells with the designed scAAV9.JeT.coGM2A vector successfully restored protein expression (FIG. 8B,C).

### Example 4: A Small Short-Term In Vivo Study in Murine Models of ABGM2 Showed Treatment with scAAV9.JeT.coGM2A Effectively Restored GM2A Expression and Reduced GM2 Ganglioside Accumulation in Comparison to Untreated Controls

To confirm in vitro results, in vivo experiments were performed with GM2A vectors. The scAAV9.JeT.coGM2A vector as described above was delivered to Neu3−/− Gm2a−/− mice. As a comparison, the scAAV9.CBh.wtGM2A vector was used which is similar but under the control of the CBh promoter instead of the JeT promoter.

### GM2A Expression was Restored in the Liver and CNS of Treated Animals of ABGM2.

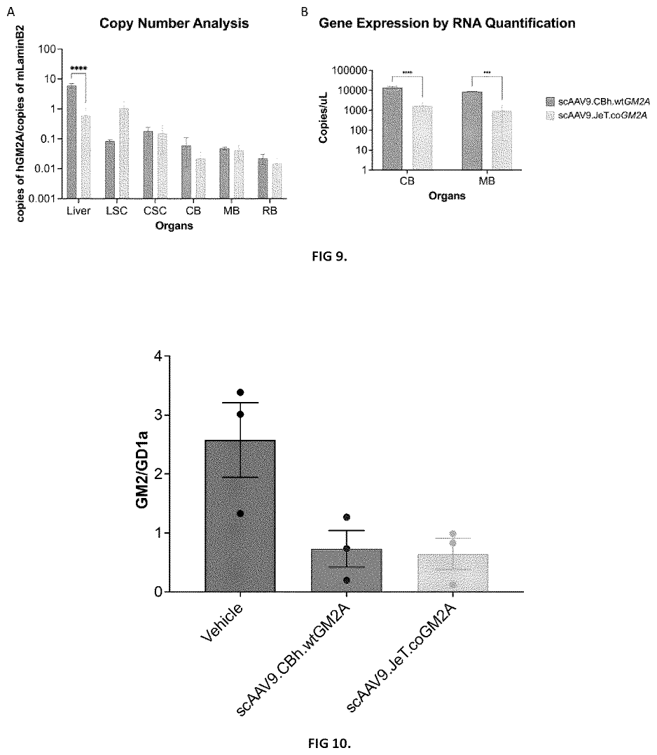

Vector biodistribution was conducted on all portions of the CNS and liver. GM2A was detectable in in all three regions of the brain, the spinal cord, and the liver (FIG. 9A). Significantly more GM2A was detected in the liver when the Cbh promoter was used versus the JeT promoter. The copy number was similar between vectors in all areas of the CNS, except the lumbar spinal cord (FIG. 9A).

RNA was extracted from liver and brain tissue (caudal- and mid-section) following euthanization of all mice in the study as described above. GM2A expression from both vectors was detected in both the caudal-section (cerebellum) and mid-section (cortex) of the brain. However, GM2A expression from the scAAV9.JeT.coGM2A vector was significantly lower than GM2A expression from the scAAV9.CBh.wtGM2A vector (FIG. 9B). This was indicative of successful gene delivery of GM2A by the SCAAV9.JeT.coGM2A vector.

### GM2 Ganglioside Accumulation was Reduced in Treated Mice Compared to Untreated Controls.

GM2 ganglioside accumulation was assessed in the mid-section of the brain. GM2 accumulation was non-significantly decreased in animals treated with both vectors (FIG. 10). Although, there was a noticeable decrease in accumulation. While the scAAV9.CBh.wtGM2A vector had higher expression, scAAV9.JeT.coGM2A appeared to function similarly in terms of GM2 metabolism.

While the present disclosure has been described with reference to examples, it is to be understood that the scope of the claims should not be limited by the embodiments set forth in the examples but should be given the broadest interpretation consistent with the description as a whole.

### Sequences:

SEQ ID NO: 1: Codon Optimized GM2A Sequence
ATGCAAAGCCTGATGCAGGCTCCTCTGCTGATCGCCCTGGGACTGCTGCTCGCCG
CCCCTGCCCAGGCCCACCTGAAGAAACCTAGCCAGCTGTCTAGCTTTAGCTGGGA
CAATTGCGACGAGGGCAAGGACCCCGCCGTGATCAGAAGCCTTACACTGGAACCT
GACCCTATCATCGTGCCTGGCAACGTGACCCTGTCCGTCATGGGCTCTACAAGCG
TTCCTCTGAGCAGCCCTCTGAAGGTGGACCTGGTGCTGGAGAAGGAGGTGGCCG
GACTGTGGATCAAGATTCCTTGTACCGATTATATCGGCTCTTGTACCTTCGAGCACT
TCTGCGATGTGCTGGATATGCTGATCCCCACCGGCGAGCCTTGCCCGGAACCCCT
GAGAACATACGGCCTGCCATGCCACTGCCCCTTCAAGGAAGGAACCTACTCCCTG
CCTAAGAGCGAGTTCGTGGTGCCCGACCTGGAACTGCCAAGTTGGCTGACAACCG
GCAACTACAGAATCGAGTCCGTGCTGAGCAGCAGCGGCAAGCGGCTGGGCTGCA
TCAAGATCGCCGCTTCTCTGAAAGGCATCTGA
SEQ ID NO: 2: GM2A Protein Sequence (translation of SEQ ID NO: 1)
MQSLMQAPLLIALGLLLAAPAQAHLKKPSQLSSFSWDNCDEGKDPAVIRSLTLEPDPIIV
PGNVTLSVMGSTSVPLSSPLKVDLVLEKEVAGLWIKIPCTDYIGSCTFEHFCDVLDMLIP
TGEPCPEPLRTYGLPCHCPFKEGTYSLPKSEFVVPDLELPSWLTTGNYRIESVLSSSGK
RLGCIKIAASLKGI*
* = stop codon
SEQ ID NO: 3: Full Sequence GM2A including pUC57 Kan
TCGCGCGTTTCGGTGATGACGGTGAAAACCTCTGACACATGCAGCTCCCGGAGAC
GGTCACAGCTTGTCTGTAAGCGGATGCCGGGAGCAGACAAGCCCGTCAGGGCGC
GTCAGCGGGTGTTGGCGGGTGTCGGGGCTGGCTTAACTATGCGGCATCAGAGCA
GATTGTACTGAGAGTGCACCATATGCGGTGTGAAATACCGCACAGATGCGTAAGG
AGAAAATACCGCATCAGGCGCCATTCGCCATTCAGGCTGCGCAACTGTTGGGAAG
GGCGATCGGTGCGGGCCTCTTCGCTATTACGCCAGCTGGCGAAAGGGGGATGTG
CTGCAAGGCGATTAAGTTGGGTAACGCCAGGGTTTTCCCAGTCACGACGTTGTAAA
ACGACGGCCAGAGAATTCGAGCTCGGTACCTCGCGAATACATCTAGATGGCCACT
CCCTCTCTGCGCGCTCGCTCGCTCACTGAGGCCGGGCGACCAAAGGTCGCCCGA
CGCCCGGGCTTTGCCCGGGCGGCCTCAGTGAGCGAGCGAGCGCGCAGAGAGGG
AGTGGCCAACTCCATCACTAGGGGTTCCTGGAGGGGTGGAGTCGTGAGCAGAATT
CGCCGGGCGGAGTTAGGGCGGAGCCAATCAGCGTGCGCCGTTCCGAAAGTTGCC
TTTTATGGCTGGGCGGAGAATGGGCGGTGAACGCCGATGATTATATAAGGACGCG
CCGGGTGTGGCACAGCTAGTTCCGTCGCAGCCGGGATTTGGGTCGCGGTTCTTGT
TTGTTCCGGAAAGCCACCATGCAAAGCCTGATGCAGGCTCCTCTGCTGATCGCCC
TGGGACTGCTGCTCGCCGCCCCTGCCCAGGCCCACCTGAAGAAACCTAGCCAGCT
GTCTAGCTTTAGCTGGGACAATTGCGACGAGGGCAAGGACCCCGCCGTGATCAGA
AGCCTTACACTGGAACCTGACCCTATCATCGTGCCTGGCAACGTGACCCTGTCCGT
CATGGGCTCTACAAGCGTTCCTCTGAGCAGCCCTCTGAAGGTGGACCTGGTGCTG
GAGAAGGAGGTGGCCGGACTGTGGATCAAGATTCCTTGTACCGATTATATCGGCT
CTTGTACCTTCGAGCACTTCTGCGATGTGCTGGATATGCTGATCCCCACCGGCGA
GCCTTGCCCGGAACCCCTGAGAACATACGGCCTGCCATGCCACTGCCCCTTCAAG
GAAGGAACCTACTCCCTGCCTAAGAGCGAGTTCGTGGTGCCCGACCTGGAACTGC
CAAGTTGGCTGACAACCGGCAACTACAGAATCGAGTCCGTGCTGAGCAGCAGCGG
CAAGCGGCTGGGCTGCATCAAGATCGCCGCTTCTCTGAAAGGCATCTGAGTCGAC
GCCAATAAAGAGCTCAGATGCATCGATCAGAGTGTGTTGGTTTTTTGTGTGCCACT
CCCTCTCTGCGCGCTCGCTCGCTCACTGAGGCCGGGCGACCAAAGGTCGCCCGA
CGCCCGGGCTTTGCCCGGGCGGCCTCAGTGAGCGAGCGAGCGCGCAGAGAGGG
AATCGGATCCCGGGCCCGTCGACTGCAGAGGCCTGCATGCAAGCTTGGTGTAATC
ATGGTCATAGCTGTTTCCTGTGTGAAATTGTTATCCGCTCACAATTCCACACAACAT
ACGAGCCGGAAGCATAAAGTGTAAAGCCTGGGGTGCCTAATGAGTGAGCTAACTC
ACATTAATTGCGTTGCGCTCACTGCCCGCTTTCCAGTCGGGAAACCTGTCGTGCCA
GCTGCATTAATGAATCGGCCAACGCGCGGGGAGAGGCGGTTTGCGTATTGGGCG
CTCTTCCGCTTCCTCGCTCACTGACTCGCTGCGCTCGGTCGTTCGGCTGCGGCGA
GCGGTATCAGCTCACTCAAAGGCGGTAATACGGTTATCCACAGAATCAGGGGATAA
CGCAGGAAAGAACATGTGAGCAAAAGGCCAGCAAAAGGCCAGGAACCGTAAAAAG
GCCGCGTTGCTGGCGTTTTTCCATAGGCTCCGCCCCCCTGACGAGCATCACAAAA
ATCGACGCTCAAGTCAGAGGTGGCGAAACCCGACAGGACTATAAAGATACCAGGC
GTTTCCCCCTGGAAGCTCCCTCGTGCGCTCTCCTGTTCCGACCCTGCCGCTTACC
GGATACCTGTCCGCCTTTCTCCCTTCGGGAAGCGTGGCGCTTTCTCATAGCTCACG
CTGTAGGTATCTCAGTTCGGTGTAGGTCGTTCGCTCCAAGCTGGGCTGTGTGCAC
GAACCCCCCGTTCAGCCCGACCGCTGCGCCTTATCCGGTAACTATCGTCTTGAGT
CCAACCCGGTAAGACACGACTTATCGCCACTGGCAGCAGCCACTGGTAACAGGAT
TAGCAGAGCGAGGTATGTAGGCGGTGCTACAGAGTTCTTGAAGTGGTGGCCTAAC
TACGGCTACACTAGAAGAACAGTATTTGGTATCTGCGCTCTGCTGAAGCCAGTTAC
CTTCGGAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACTCACGTT
AAGGGATTTTGGTCATGAGATTATCAAAAAGGATCTTCACCTAGATCCTTTTAAATT
AAAAATGAAGTTTTAAATCAAGCCCAATCTGAATAATGTTACAACCAATTAACCAATT
CTGATTAGAAAAACTCATCGAGCATCAAATGAAACTGCAATTTATTCATATCAGGAT
TATCAATACCATATTTTTGAAAAAGCCGTTTCTGTAATGAAGGAGAAAACTCACCGA
GGCAGTTCCATAGGATGGCAAGATCCTGGTATCGGTCTGCGATTCCGACTCGTCC
AACATCAATACAACCTATTAATTTCCCCTCGTCAAAAATAAGGTTATCAAGTGAGAA
ATCACCATGAGTGACGACTGAATCCGGTGAGAATGGCAAAAGTTTATGCATTTCTTT
CCAGACTTGTTCAACAGGCCAGCCATTACGCTCGTCATCAAAATCACTCGCATCAA
CCAAACCGTTATTCATTCGTGATTGCGCCTGAGCGAGACGAAATACGCGATCGCTG
TTAAAAGGACAATTACAAACAGGAATCGAATGCAACCGGCGCAGGAACACTGCCA
GCGCATCAACAATATTTTCACCTGAATCAGGATATTCTTCTAATACCTGGAATGCTG
TTTTTCCGGGGATCGCAGTGGTGAGTAACCATGCATCATCAGGAGTACGGATAAAA
TGCTTGATGGTCGGAAGAGGCATAAATTCCGTCAGCCAGTTTAGTCTGACCATCTC
ATCTGTAACATCATTGGCAACGCTACCTTTGCCATGTTTCAGAAACAACTCTGGCG
CATCGGGCTTCCCATACAAGCGATAGATTGTCGCACCTGATTGCCCGACATTATCG
CGAGCCCATTTATACCCATATAAATCAGCATCCATGTTGGAATTTAATCGCGGCCTC
GACGTTTCCCGTTGAATATGGCTCATAACACCCCTTGTATTACTGTTTATGTAAGCA
GACAGTTTTATTGTTCATGATGATATATTTTTATCTTGTGCAATGTAACATCAGAGAT
TTTGAGACACGGGCCAGAGCTGCA
SEQ ID NO: 4: 5′ ITR
GGCCACTCCCTCTCTGCGCGCTCGCTCGCTCACTGAGGCCGGGCGACCAAAGGT
CGCCCGACGCCCGGGCTTTGCCCGGGCGGCCTCAGTGAGCGAGCGAGCGCGCA
GAGAGGGAGTGGCCAACTCCATCACTAGGGGTTCCTGGAGGGGTGGAGTCGTGA
SEQ ID NO: 5: 3′ Truncated ITR
CCACTCCCTCTCTGCGCGCTCGCTCGCTCACTGAGGCCGGGCGACCAAAGGTCG
CCCGACGCCCGGGCTTTGCCCGGGCGGCCTCAGTGAGCGAGCGAGCGCGCAGA
GAGGGA
SEQ ID NO: 6: Spacer + JeT promoter + Kozak sequence
GCAGAATTCGCCGGGCGGAGTTAGGGCGGAGCCAATCAGCGTGCGCCGTTCCGA
AAGTTGCCTTTTATGGCTGGGCGGAGAATGGGCGGTGAACGCCGATGATTATATAA
GGACGCGCCGGGTGTGGCACAGCTAGTTCCGTCGCAGCCGGGATTTGGGTCGCG
GTTCTTGTTTGTTCCGGAAAGCCACC
SEQ ID NO: 7: Spacer + PolyA Region
GTCGACGCCAATAAAGAGCTCAGATGCATCGATCAGAGTGTGTTGGTTTTTTGTGT
G

### REFERENCES

1. Bley, A. E., Giannikopoulos, O. A., Hayden, D., Kubilus, K., Tifft, C. J., & Eichler, F. S. (2011). Natural History of Infantile GM2 Gangliosidosis. Pediatrics, 128(5), e1233-e1241. https://doi.org/10.1542/peds.2011-0078
2. Cachón-González, M. B., Zaccariotto, E., & Cox, T. M. (2018). Genetics and Therapies for GM2 Gangliosidosis. Current Gene Therapy, 18(2), 68-89. https://doi.org/10.2174/1566523218666180404162622
3. Cachón-González, M. B., Wang, S. Z., Lynch, A., Ziegler, R., Cheng, S. H., & Cox, T. M. (2006). Effective gene therapy in an authentic model of Tay-Sachs-related diseases. Proceedings of the National Academy of Sciences, 103(27), 10373-10378. https://doi.org/10.1073/pnas.0603765103
4. Ornaghi, F., Sala, D., Tedeschi, F., Maffia, M. C., Bazzucchi, M., Morena, F., Valsecchi, M., Aureli, M., Martino, S., & Gritti, A. (2020). Novel bicistronic lentiviral vectors correct β-Hexosaminidase deficiency in neural and hematopoietic stem cells and progeny: Implications for in vivo and ex vivo gene therapy of GM2 gangliosidosis. Neurobiology of Disease, 134, 104667. https://doi.org/10.1016/j.nbd.2019.104667
5. Osmon, K. J. L., Woodley, E., Thompson, P., Ong, K., Karumuthil-Melethil, S., Keimel, J. G., Mark, B. L., Mahuran, D., Gray, S. J., & Walia, J. S. (2016). Systemic Gene Transfer of a Hexosaminidase Variant Using an scAAV9.47 Vector Corrects GM2 Gangliosidosis in Sandhoff Mice. Human Gene Therapy, 27(7), 497-508. https://doi.org/10.1089/hum.2016.015
6. Lundstrom, K. (2018). Viral Vectors in Gene Therapy. Diseases, 6(2), Article 2. https://doi.org/10.3390/diseases6020042
7. Saraiva, J., Nobre, R. J., & Pereira de Almeida, L. (2016). Gene therapy for the CNS using AAVs: The impact of systemic delivery by AAV9. Journal of Controlled Release, 241, 94-109. https://doi.org/10.1016/j.jconrel.2016.09.011
8. Leone, P., Shera, D., McPhee, S. W. J., Francis, J. S., Kolodny, E. H., Bilaniuk, L. T., Wang, D.-J., Assadi, M., Goldfarb, O., Goldman, H. W., Freese, A., Young, D., During, M. J., Samulski, R. J., & Janson, C. G. (2012). Long-Term Follow-Up After Gene Therapy for Canavan Disease. Science Translational Medicine, 4(165), 165ra163-165ra163. https://doi.org/10.1126/scitranslmed.3003454
9. Shirley, J. L., de Jong, Y. P., Terhorst, C., & Herzog, R. W. (2020). Immune Responses to Viral Gene Therapy Vectors. Molecular Therapy, 28(3), 709-722. https://doi.org/10.1016/j.ymthe.2020.01.001
10. McCarty, D. M., Young, S. M., & Samulski, R. J. (2004). Integration of Adeno-Associated Virus (AAV) and Recombinant AAV Vectors. Annual Review of Genetics, 38(1), 819-845. https://doi.org/10.1146/annurev.genet.37.110801.143717
11. Duque, S., Joussemet, B., Riviere, C., Marais, T., Dubreil, L., Douar, A.-M., Fyfe, J., Moullier, P., Colle, M.-A., & Barkats, M. (2009). Intravenous Administration of Self-complementary AAV9 Enables Transgene Delivery to Adult Motor Neurons. Molecular Therapy, 17(7), 1187-1196. https://doi.org/10.1038/mt.2009.71
12. Foust, K. D., Nurre, E., Montgomery, C. L., Hernandez, A., Chan, C. M., & Kaspar, B. K. (2009). Intravascular AAV9 preferentially targets neonatal neurons and adult astrocytes. Nature Biotechnology, 27(1), Article 1. https://doi.org/10.1038/nbt. 1515
13. Tornoe, J. (2002). JeT promoter (United States Patent No. US20020098547A1). https://patents.google.com/patent/US20020098547A1/en
14. Bailey, R. M., Armao, D., Nagabhushan Kalburgi, S., & Gray, S. J. (2018). Development of Intrathecal AAV9 Gene Therapy for Giant Axonal Neuropathy. Molecular Therapy—Methods & Clinical Development, 9, 160-171. https://doi.org/10.1016/j.omtm.2018.02.005
15. Mussche, S., Devreese, B., Nagabhushan Kalburgi, S., Bachaboina, L., Fox, J. C., Shih, H.-J., Van Coster, R., Samulski, R. J., & Gray, S. J. (2013). Restoration of Cytoskeleton Homeostasis After Gigaxonin Gene Transfer for Giant Axonal Neuropathy. Human Gene Therapy, 24(2), 209-219. https://doi.org/10.1089/hum.2012.107
16. Athey, J., Alexaki, A., Osipova, E., Rostovtsev, A., Santana-Quintero, L. V., Katneni, U., Simonyan, V., & Kimchi-Sarfaty, C. (2017). A new and updated resource for codon usage tables. BMC Bioinformatics, 18(1), 391. https://doi.org/10.1186/s12859-017-1793-7
17. Mauro, V. P., & Chappell, S. A. (2014). A critical analysis of codon optimization in human therapeutics. Trends in Molecular Medicine, 20(11), 604-613. https://doi.org/10.1016/j.molmed.2014.09.003
18. Bauer, A. P., Leikam, D., Krinner, S., Notka, F., Ludwig, C., Längst, G., & Wagner, R. (2010). The impact of intragenic CpG content on gene expression. Nucleic Acids Research, 38(12), 3891-3908. https://doi.org/10.1093/nar/gkq115
19. Kudla, G., Lipinski, L., Caffin, F., Helwak, A., & Zylicz, M. (2006). High Guanine and Cytosine Content Increases mRNA Levels in Mammalian Cells. PLOS Biology, 4(6), e180. https://doi.org/10.1371/journal.pbio.0040180
20. Newman, Z. R., Young, J. M., Ingolia, N. T., & Barton, G. M. (2016). Differences in codon bias and GC content contribute to the balanced expression of TLR7 and TLR9. Proceedings of the National Academy of Sciences, 113(10), E1362-E1371. https://doi.org/10.1073/pnas. 1518976113
21. Alexaki, A., Hettiarachchi, G. K., Athey, J. C., Katneni, U. K., Simhadri, V., Hamasaki-Katagiri, N., Nanavaty, P., Lin, B., Takeda, K., Freedberg, D., Monroe, D., McGill, J. R., Peters, R., Kames, J. M., Holcomb, D. D., Hunt, R. C., Sauna, Z. E., Gelinas, A., Janjic, N., . . . . Kimchi-Sarfaty, C. (2019). Effects of codon optimization on coagulation factor IX translation and structure: Implications for protein and gene therapies. Scientific Reports, 9(1), Article 1. https://doi.org/10.1038/s41598-019-51984-2
22. McCarty, D. M., Fu, H., Monahan, P. E., Toulson, C. E., Naik, P., and Samulski, R. J. (2003). Adeno-associated virus terminal repeat (TR) mutant generates self-complementary vectors to overcome the rate-limiting step to transduction in vivo. Gene Therapy 10, 2112-2118.
23. Bailey, R. M., Rozenberg, A., and Gray, S. J. (2020). Comparison of high-dose intracisterna magna and lumbar puncture intrathecal delivery of AAV9 in mice to treat neuropathies. Brain Research 1739, 146832.
24. Gray, S. J., Choi, V. W., Asokan, A., Haberman, R. A., Mccown, T. J., and Samulski, R. J. (2011). Production of Recombinant Adeno-Associated Viral Vectors and Use in In Vitro and In Vivo Administration. Curr Protoc Neurosci CHAPTER, Unit4.17.
25. Glombitza, G. J., Becker, E., Kaiser, H. W., and Sandhoff, K. (1997). Biosynthesis, processing, and intracellular transport of GM2 activator protein in human epidermal keratinocytes. The lysosomal targeting of the GM2 activator is independent of a mannose-6-phosphate signal. Journal of Biological Chemistry 272 (8), 5199-5207.

1. A nucleic acid construct comprising a nucleotide sequence encoding a GM2 activator (GM2A) protein operably linked to a promoter and a transcription termination site.

2. The nucleic acid construct of claim 1, wherein the nucleotide sequence encoding the GM2A protein is set forth in SEQ ID NO: 1, or a functional variant thereof.

3. The nucleic acid construct of claim 1, wherein the GM2A protein has an amino acid sequence that is at least 50%, at least 60%, at least 70%, at least 80%, at least 90%, at least 95%, or at least 99% identical to the protein encoded by SEQ ID NO: 1, and which retains GM2A activity.

4. The nucleic acid construct according to claim 1, encoding a GM2A protein comprising an amino acid sequence set forth in SEQ ID NO: 2, or a functional variant thereof.

5. The nucleic acid construct according to claim 1, wherein the promoter is a constitutive promoter or a tissue- or cell-specific promoter.

6. The nucleic acid construct according to claim 5, wherein the promoter is selected from the group consisting of: chicken beta actin (CBA), chicken β-actin hybrid (CBh), CMV early enhancer (CAG), Elongation Factor 1α (eF-1a), simian virus 40 early promoter (SV40), human phosphoglycerate kinase 1 (PGK), cytomegalovirus immediate-early promoter (CMV), human β-actin (hACTB) and JeT synthetic promoter.

7. The nucleic acid construct of claim 1, comprising a sequence set forth in SEQ ID NO: 3, or a functional variant thereof.

8. A viral vector comprising the nucleic acid construct according to claim 1.

9. The viral vector according to claim 8, wherein the viral vector is an Adeno-Associated Virus (AAV) vector or a derivative thereof.

10. The viral vector according to claim 9, wherein the AAV vector is selected from the group consisting of: AAV1, AAV2, AAV5, AAV6, AAV7, AAV8, AAV9, AAVrh10 and derivatives thereof.

11. The viral vector according to claim 9 wherein the viral vector is a chimeric, shuffled or capsid modified derivative of AAV.

12. A pharmaceutical composition comprising the nucleic acid construct of claim 1, and a pharmaceutically acceptable carrier or diluent.

13. The pharmaceutical composition of claim 12, wherein the pharmaceutically acceptable carrier or diluent is a lipid nanoparticle.

14. The pharmaceutical composition of claim 12, wherein the pharmaceutical composition is formulated for intravenous or intrathecal administration.

15-19. (canceled)

20. A kit comprising the nucleic acid construct of claim 1, and instructions for use thereof.

21. A method of treating or preventing AB-variant GM2 Gangliosidosis (ABGM2) in a subject in need thereof, comprising administering to the subject a therapeutically effective amount of the viral vector of claim 8.

22. The method of claim 21, wherein the administering is by intravenous and/or intrathecal injection.

23. A pharmaceutical composition comprising the viral vector of claim 8 and a pharmaceutically acceptable carrier or diluent.

24. The pharmaceutical composition of claim 23, wherein the pharmaceutically acceptable carrier or diluent is a lipid nanoparticle.

25. The pharmaceutical composition of claim 23, wherein the pharmaceutical composition is formulated for intravenous or intrathecal administration.
